(19) United States
(12) Patent Application Publication
(10) Pub. No.: US 2023/0346783 A1
(43) Pub. Date: Nov. 2, 2023

(54) THERAPEUTIC STRATEGIES TO MANAGE FACIAL CONTRACTURES POST INJURY

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Mohamed EL MASRY, Indianapolis, IN (US); Vinoj GOPALAKRISHNAN, Indianapolis, IN (US)

(21) Appl. No.: 17/914,587

(22) PCT Filed: Apr. 7, 2021
(86) PCT No.: PCT/US2021/026214

§ 371 Date: Sep. 26, 2022

Related U.S. Application Data
(60) Provisional application No. 63/007,101, filed on Apr. 8, 2020.

(51) Int. Cl. A61K 31/506; A61K 45/06; A61P 17/02

(52) U.S. Cl. CPC A61K 31/506; A61K 45/06; A61P 17/02

(57) ABSTRACT

Compositions and methods are provided for reducing facial contracture and to alleviate deficits associated with thermal injury of the face, including ectropion (epithelial-ocular junction), eversion of the lip (epithelial-oral junction), and oral incompetence (drooling). In accordance with one embodiment such compositions are used in conjunction with known treatments for use on burn patients.

### CROSS REFERENCE TO RELATED APPLICATIONS

This application claims priority to U.S. Provisional Patent Application No. 63/007,101 filed on Apr. 8, 2020, the disclosure of which is expressly incorporated herein.

### BACKGROUND OF THE DISCLOSURE

The face is often the seat of recognition for a human being, It is perhaps the most powerful “channel” of nonverbal communication and living with a distortion in the appearance of one's face as a result of injury due to burns or other trauma is challenging. In the United States, about 1.25 million people are treated each year for burns, and 40,000 are hospitalized for the treatment of these injuries resulting in high medical costs of approximately $7.9 billion per year. One source of maxillofacial thermal injuries arises from exposure to the heat of an explosive blast that causes flash burn of an unprotected face. These burns involve the skin, underlying muscle and often even the bone. Because of the high heat involved in explosive blasts, sixth degree burns are common. Maxillofacial thermal or other injuries cause major contractures viewed as severe scarring that burdens the subject socially, emotionally, psychologically and functionally. Critical and unique functional deficits in thermal injury of the face induced by such skin contracture include ectropion (epithelial-ocular junction), eversion of the lip (epithelial-oral junction) and oral incompetence (drooling).

Facial skin, unlike skin of the rest of the body, is of neural crest origin (as opposed to mesodermal origin in other parts of the body). Given this difference, the development of facial scar was investigated and found by applicant to follow a mechanistically unique path where contractile elements of the skeletal (mostly) and cardiac (few) muscles are expressed in the skin. Compared to scar response in other parts of the body, facial scar contractures are much more severe. Existing scar therapies originate from studies on skin of mesodermal origin and are not beneficial to applications in the context of facial contractures. Given the lack of appropriate face burn models, to test therapies, this difference could not be previously distinguished. Accordingly the present disclosure is directed to novel methods for treating injury to facial skin resulting from burns or other trauma. The methods could potentially be extended to manage contractures in other parts of the body.

### SUMMARY

In accordance with one embodiment of the present disclosure, a method of treating patients suffering from the effects of maxillofacial injuries is provided, including for example, maxillofacial thermal injuries that result in severe facial contractures that cause disfiguration. Applicant has discovered that facial contractures follow a different pathway than scar development in other skin tissue on the body. This alternate pathways leads to facial scar contractures being much more severe compared to scar response in other parts of the body. In damaged facial skin, contractile elements of the skeletal (mostly) and cardiac (few) muscles are expressed in the cells involved in repairing the damage tissue, leading to severe contractures in the resultant scar tissue. Accordingly, facial scars are more intractable to current strategies for treating scars.

As disclosed herein, applicant's discovery that contractile elements are induced in damaged facial skin led to applicant' proposed novel strategies of treating both existing mature scar (late stage), and for minimizing post injury facial contracture formation (early stage). The methods disclosed herein can reduce or prevent contractures associated with existing facial scars and alleviate deficits associated with thermal injury of the face, including reducing ectropion (epithelial-ocular junction), eversion of the lip (epithelial-oral junction) and oral incompetence (drooling).

As disclosed herein drugs that target muscle relaxation are anticipated to help treat mature facial contractures. In early stage, shortly after the injury (two weeks to 2 months) drugs inhibiting myogenesis will help. Examples of late stage FDA-approved muscle relaxer drugs that can be repurposed include: baclofen, dantrolene, and tizanidine. Additional muscle relaxer drugs suitable for use in the present invention include carisoprodol, chlorzoxazone, cyclobenzaprine, metaxalone, methocarbamol, and orphenadrine. Examples of early stage repurposed myogenesis inhibitor drugs include Nilotinib (Tasigna/AMN107®; Novartis). Also, other inhibitors of myogenesis such as but not limited to stromal interaction molecule (STIM) inhibitors and estrogen-related receptor alpha inhibitors are suitable for use in the present invention.

In accordance with one embodiment a method of treating mature scar tissue located on the face or neck of mammalian subject (optionally a human subject) is provided, wherein a composition comprising a skeletal muscle relaxant is administered to a subject in need of such treatment. In one embodiment the skeletal muscle relaxant is selected from the group consisting of baclofen, dantrolene, tizanidine. carisoprodol, chlorzoxazone, cyclobenzaprine, metaxalone, methocarbamol, and orphenadrine. In one embodiment a method is provided for reducing the severity of scar tissue formation on the face or neck of mammalian subject, optionally a human subject, during the healing phase after injury, wherein the method comprises the step of administering a composition comprising a myogenesis inhibitor to a subject in need of such treatment. In one embodiment the myogenesis inhibitor is Nilotinib (Tasigna/AMN107®; Novartis).

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans and includes individuals not under the direct care of a physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein a reference to a “face” includes the front part of the head that in humans extends from the forehead to the chin and extends laterally to the sides of the head and includes the ears, mouth, nose, cheeks, and eyes.

As used herein “facial trauma”, also called maxillofacial trauma, is any physical trauma to the face and the neck. Facial trauma can involve soft tissue injuries such as burns, lacerations and bruises, and may include fractures and damage to facial bones such as nasal fractures and fractures of the jaw.

As defined herein “wound healing” defines a process wherein a living organism replaces destroyed or damaged tissue by newly produced tissue.

### Embodiments

Scars form as part of the natural healing process when the dermis is damaged. In response to such damage, the body forms new collagen fibers to mend the damage, resulting in the formation of scar tissue. Scar tissue, if treated appropriately, can typically be remodeled to resemble normal, healthy tissue. However, depending on the extent of the initial injury and healing responses some scars are severe and can be disfiguring.

For example, the heat of an explosive blast causes flash burn of the face, not protected by armor. These burns involve the skin, underlying muscle and often even the bone. Because of the high heat involved, fourth to sixth degree burns are common. Such maxillofacial thermal injuries cause major facial contractures that burdens the subject socially, emotionally, psychologically and functionally. Critical and unique functional deficits in thermal injury of the face include ectropion (epithelial-ocular junction), eversion of the lip (epithelial-oral junction) and oral incompetence (drooling). Compared to scar response in other parts of the body, facial contractures are much more severe.

To develop novel strategies to manage facial contractures, the molecular mechanisms of post-burn facial contractures versus scarring of skin of the back were compared in a pair-matched pre-clinical porcine setting. Facial skin, unlike skin of the rest of the body, is of neural crest origin. Sequencing studies on a severe face burn model led to the discovery that myogenic genes play a unique role in contracture formation in skin tissue of the face. More particularly, applicant has discovered that following a severe burn, skin and related wound cells in the face undergo myogenic conversion resulting in overt contracture that is responsible for the unique characteristics in thermal injury of the face described above. Repair of skin tissue of the face follows a mechanistically unique path where contractile elements of the skeletal (mostly) and cardiac (few) muscles are expressed in the skin. Scarring of the face cannot be readily treated by conventional anti-scarring strategies currently used today. In accordance with the present disclosure the treatment of facial contractures can be managed using two approaches based on the stage of facial wound repair. One treatment strategy relates to existing mature scar (late stage), where existing scars are treated to alleviate pain and disfigurement associated with the scars. Another method of treatment relates to post injury contractures formation (early stage) and the administration of therapeutics to diminish the formation of scar tissue including decreasing the amount of collagen present in the scar tissue that forms and/or decreasing surface area of the scar tissue.

In accordance with one embodiment drugs that stimulate muscle relaxation will be used to treat mature facial contractures. In one embodiment the subject is a human and the mature facial contracture to be treated is a severe scar resulting from traumatic damage to facial skin. In one embodiment the scar is formed after healing from a burn caused by tissue damage caused by heat, chemicals, electricity, sunlight, or nuclear radiation. In one embodiment the method of the present disclosure is directed to treating mature contractures formed after thermal damage to human facial skin, including scars formed after healing from second, third, fourth, fifth or sixth degree burns.

One method of treating mature contractures located on the face or neck of mammalian subject comprises the step of administering a composition comprising a skeletal muscle relaxant and a pharmaceutically acceptable carrier to a subject in need of such treatment. In accordance with one embodiment a pharmaceutical formulation is prepared comprising a compound selected from the group consisting of baclofen, dantrolene, tizanidine, carisoprodol, chlorzoxazone, cyclobenzaprine, metaxalone, methocarbamol, and orphenadrine. In one embodiment the pharmaceutical composition is formulated for administration by any acceptable route including as an oral, injectable or topical formulation. In one embodiment the composition is formulated for oral administration. In one embodiment the composition is formulated as a topical cream or ointment that is placed in contact with the external surface of the scar. In one embodiment the topical cream or ointment can be directly applied to the scar tissue, or the scar can be covered in gauze, bandage or other matrix that releases the formulation to the patient's affected skin a time released manner.

In one embodiment a method of reducing the severity of scar tissue formation during the process of healing after a traumatic injury to the face of mammalian subject is provided. In one embodiment the method comprises the step of administering a composition comprising a myogenesis inhibitor to a subject in need of such treatment. In one embodiment the method comprises treating a human recovering from facial burns to reduce or prevent the amount of scar tissue formation during the healing process, wherein a pharmaceutical composition comprising a myogenesis inhibitor and a pharmaceutically acceptable carrier is administered to the patient. Reducing the amount of scar tissue formation includes any one of reducing the amount of contracture in the formed scar tissue, reducing the amount of collagen in the formed scar tissue, reducing the size of the scar. In one embodiment the myogenesis inhibitor is Nilotinib. In one embodiment the pharmaceutical composition comprises a myogenesis inhibitor as well as a skeletal muscle relaxant, including a muscle relaxant selected from the group consisting of baclofen, dantrolene, tizanidine. carisoprodol, chlorzoxazone, cyclobenzaprine, metaxalone, methocarbamol, and orphenadrine. In one embodiment the pharmaceutical compositions of the present invention are administered 2, 3, 4, 5, 6 weeks after the initial trauma to the face.

In one embodiment a pharmaceutical composition for reducing the severity of scar tissue formation during the process of healing after a traumatic injury to the face is formulated for administration by any acceptable route including as an oral, injectable or topical formulation. In one embodiment the composition is formulated for oral administration. In one embodiment the composition is formulated as a topical cream or ointment that is placed in contact with the external surface of the scar. In one embodiment the topical cream or ointment can be directly applied to the scar tissue or the scar can be covered in gauze, bandage or other matrix that releases the formulation to the patient's affected skin in a time released manner In one embodiment a composition comprising a myogenesis inhibitor and a skeletal muscle relaxant is formulated as a cream, ointment or time release matrix to be directly applied to facial contracture tissue formed, or in the process of being formed, after thermal trauma to facial skin.

In accordance with the present disclosure compositions comprising a myogenesis inhibitor and/or a skeletal muscle relaxant can be used to treating patients suffering from maxillofacial thermal injuries. The methods disclosed herein reduce or prevent facial contracture formation to alleviate deficits associated with thermal injury of the face, including ectropion (epithelial-ocular junction), eversion of the lip (epithelial-oral junction) and oral incompetence (drooling). In accordance with one embodiment such compositions are used in conjunction with known treatments for use on burn patients, including dermabrasion, chemical peels and laser resurfacing and it is not a replacement of any indicated surgical reconstruction required.

In one embodiment a composition is provided for treating facial burns, wherein the composition comprises a myogenesis inhibitor, a skeletal muscle relaxant and a pharmaceutically acceptable carrier. In one embodiment the composition is formulated for topical application, including for example as a gel, ointment, lotion or cream. The topical formulation may include water, oil, alcohol or propylene glycol mixed with preservatives, emulsifiers, or absorption promoters. In one embodiment the composition is prepared as a gel or other matrix that releases the active agent in a time release manner In one embodiment the composition is prepared as a transdermal patch or bandage that is applied to the scar.

### EXAMPLE 1

### Wound Healing in Porcine Maxillo-Facial Thermal Trauma Model

### Materials and Methods

White pigs were subjected to severe burn trauma in the facial area (˜50% of the face) or on dorsum 6 (2″×2″) burn wounds. Progression of burn wound healing were followed till day 84 using non-invasive imaging: a) laser speckle microperfusion imaging (LSI); b) harmonic ultrasound imaging with Doppler (HUSD) for tissue stiffness and blood supply; and c) computed tomography (CT) for 3D reconstruction of the facial soft tissues and bone. Additionally, wound inflammation, angiogenesis and remodeling was examined using standard immunohistochemistry. Laser capture microdissection (LCM) of the epithelium on day 84 post burn was performed followed by whole genome RNA-sequencing. CT and ultrasound imaging established sixth degree burn with bone involvement showing severe deficits including ectropion, oral eversion and contracture, excessive scarring as well as drooling during eating. All of these characteristics are consistent with manifestations commonly noted in humans with facial burn. Intense facial contracture was evident at day 84 post-burn. Vascular and bone deficits (n=7) were visualized using LSI, HUSD and CT imaging.

The specific areas of the face involving mucocutaneous junctions as opposed to scar healed in a different manner RNA seq (n=6 each group; p<0.001) and bioinformatics analysis of the LCM captured epithelium revealed differential expression of a unique sets of genes following facial burn trauma that was not present in the contracture-related genes expressed following back injury in healing epithelium. Highly significantly changes were noted in the pathways associated with contraction, morphogenesis, cytoskeleton that were uniquely upregulated in facial epithelium as compared to back.

Immunohistochemistry analysis on face scar tissues relative to back skin scar tissues revealed two proteins associated with contractile tissues (paxillin 7 (Pax7) & desmin) were highly expressed in healing dermis of the face relative to healing dermis from the back of pigs. Paxillin is a cytoskeletal protein involved in actin-membrane attachment at sites of cell adhesion to the extracellular matrix (focal adhesion) and desmin is a myofibrillar protein that is the chief intermediate filament of skeletal and cardiac muscle. Elevated expression levels of smooth muscle actin was also detected in healing epithelium of the face relative to healing epithelium from the back of pigs.

These data reveal features characteristic of human facial burns involving the mucocutaneous junctions. Histopathological analysis further revealed differential inflammatory, vascularization and scarring responses at anatomic locations. This work highlights the unique molecular signatures and pathways in facial burn contracture response that will help design adequate management strategies towards facial scar contractures.

1. A method of treating contracture tissue located on the face of a mammalian subject, said method comprising the step of administering a composition comprising a skeletal muscle relaxant to a subject in need of such treatment.

2. The method of claim 1 wherein the mammalian subject is a human.

3. The method of claim 1 wherein the muscle relaxant is a compound selected from the group consisting of baclofen, dantrolene, tizanidine, carisoprodol, chlorzoxazone, cyclobenzaprine, metaxalone, methocarbamol, and orphenadrine.

4. The method of claim 3 wherein said contractures are the result of a thermal burn.

5. The method of claim 3 wherein said composition is administered orally.

6. The method of claim 3 wherein said composition is formulated as a topical cream or ointment that is applied directly to the external surface of the contracture.

7. A method of reducing the severity of contracture on the face of a mammalian subject during the healing phase after injury to the face, said method comprising the step of administering a first composition comprising a myogenesis inhibitor to a subject in need of such treatment.

8. The method of claim 7 wherein the mammalian subject is a human.

9. The method of claim 7 further comprising the step of administering a supplemental composition comprising a skeletal muscle relaxant.

10. The method of claim 7 wherein said first composition further comprises a skeletal muscle relaxant.

11. The method of claim 7 wherein said myogenesis inhibitor is Nilotinib.

12. The method of claim 9 wherein the muscle relaxant is a compound selected from the group consisting of baclofen, dantrolene, tizanidine. carisoprodol, chlorzoxazone, cyclobenzaprine, metaxalone, methocarbamol, and orphenadrine.

13. The method of claim 12 wherein said facial injury is a facial thermal burn.

14. The method of claim 13 wherein the facial thermal burn is a third degree burn or more severe burn.

15. The method of claim 7 wherein said composition is administered orally.

16. The method of claim 7 wherein said composition is formulated as a topical cream or ointment that is applied directly to the external surface of the healing tissues of the damaged facial tissue.

17. The method of claim 10 wherein the muscle relaxant is a compound selected from the group consisting of baclofen, dantrolene, tizanidine. carisoprodol, chlorzoxazone, cyclobenzaprine, metaxalone, methocarbamol, and orphenadrine.
